(19) United States
(12) Patent Application Publication
(10) Pub. No.: US 2004/0009142 A1
(43) Pub. Date: Jan. 15, 2004

(54) Synergistically active mixture which inhibits hair growth

(76) Inventors: Marie-France Zambaux, Tomblaine (FR); Viola Hoerner-Wetzel, Seichamps (FR); Veronique Gillon, Essey-Les-Nancy (FR)

Correspondence Address: COGNIS CORPORATION, PATENT DEPARTMENT, 300 BROOKSIDE AVENUE, AMBLER, PA, 19002, US

(21) Appl. No.: 10/333,170

(22) PCT Filed: Jul. 18, 2001
(86) PCT No.: PCT/EP01/08275

(30) Foreign Application Priority Data
Jul. 26, 2000 (FR) 0009793

(51) Int. Cl. A61K 7/06; A61K 35/78

(52) U.S. Cl. 424/74; 424/730; 424/769; 424/757

(57) ABSTRACT

A process for inhibiting hair growth on skin comprising contacting the skin with a composition containing: (a) a hydrolyzed soya protein; (b) at least one plant extract selected from the group consisting of Hypericum perforatum, Hamamelis virginiana, Arnica montana, Salix alba, and mixtures thereof; and (c) optionally, an auxiliary component selected from the group consisting of urea, menthol, propylene glycol, salicylic acid, and mixtures thereof.

### FIELD OF THE INVENTION

This invention relates generally to cosmetology and/or pharmacology and, more particularly, to the use of compositions containing a synergistically acting mixture which inhibits hair growth, preferably in deodorants and/or antiperspirants or aftershave.

### PRIOR ART

Today, cosmetic preparations are available to the consumer in a variety of combinations. Consumers not only expect these cosmetics to have a certain care effect or to eliminate a certain deficiency, they are also increasingly demanding products which combine several properties and thus show an improved performance spectrum. The consumer is also entitled to expect the composition of the product to have optimal dermatological compatibility so that even sensitive consumers do not react with irritation. In addition, the preparations are also expected to perform other functions which are related increasingly to care and particularly protection. There is a particular interest in substances which represent active principles that impart desirable properties to the skin and/or hair and, at the same time, positively influence, or at least do not adversely affect, the technical properties of the cosmetic product, such as storage stability, light stability and formulatability. In addition, consumers demand high dermatological compatibility and, above all, the use of natural products. In addition, it is desirable to obtain distinctly better products by combining already known active principles or by discovering new applications for already known classes of substances. The combination of already known active principles often leads to positive synergistic effects and provides for a reduction in the concentration of the active principles to be used.

Extracts of plants and their ingredients are being increasingly used in cosmetic and pharmaceutical products. For many years, plant extracts have been used for medicinal purposes and also for cosmetic purposes in many different cultures. These plant extracts were often known only for very specific individual effects which limited their scope of application.

There is a growing interest, particularly in the borderlands between cosmetology and pharmacology, in care compositions which show pharmaceutical activity with very few side effects. If these care compositions are present in cosmetic products, the consumer is able to eliminate or prevent deficiencies conveniently and without much effort.

Apart from their principal, desired properties, special cosmetic preparations are often expected to show secondary effects which, at the same time, provide another positive care effect or inhibit unwanted effects such as, for example, vigorous growth of body hair.

Normal hair growth is generally tolerated whereas excessive hair growth is often a problem so that the hair growth has to be reduced or body parts have to be freed from hair. Various known methods are available for this purpose. Firstly, there are the mechanical methods which can be very painful and time-consuming. In addition, the small wounds formed are in danger of becoming infected.

In other known processes, the hair is removed by cold or hot wax. In their case, too, removal is only temporary and, above all, irritation can be caused.

In addition, there is shaving where the hairs are only cut off and, after a short time, grow again, generally to an increased extent. Many men and women experience skin irritation after shaving, particularly in the case of sensitive skin. Conventional aftershave preparations often sooth such irritation and refresh the skin, but do not prevent the hair from regrowing, often to an increased extent. Women in particular use shaving to remove hair inter alia from the armpits.

### DESCRIPTION OF THE INVENTION

The problem addressed by the present invention was to provide compositions which, besides showing care and protective properties, would above all be effective in inhibiting hair growth.

Another problem addressed by the invention was to provide compositions which would be effective in inhibiting hair growth and to use this effect, for example, in cosmetic and/or pharmaceutical compositions used for parts of the body where hair growth is undesirable.

The present invention relates to the use of a composition containing a synergistically active mixture which inhibits hair growth and which contains hydrolyzed soya proteins and at least one extract of a plant selected from the group consisting of Hypericum perforatum, Hamamelis virginiana, Arnica montana and Salix alba.

A preferred embodiment of the invention is the use of the above-described compositions, the synergistic mixture additionally containing substances selected from the group consisting of urea, menthol, propylene glycol and salicylic acid.

It has surprisingly been found that hair growth can be inhibited by the use of a composition containing synergistically acting mixtures for inhibiting hair growth containing hydrolyzed soya proteins and at least one extract of a plant selected from the group consisting of Hypericum perforatum, Hamamelis virginiana, Arnica montana and Salix alba and, in particular additionally, urea, menthol, propylene glycol and salicylic acid.

In a particularly preferred embodiment, the synergistic effect on the inhibition of hair growth is obtained by mixtures which contain hydrolyzed soya proteins, extracts of Hypericum perforatum, extracts of Hamamelis virginiana, extracts of Arnica montana and extracts of Salix alba and, in addition, urea, menthol, propylene glycol and salicylic acid. Accordingly, a particularly preferred embodiment is the use of compositions containing a synergistically acting mixture for inhibiting hair growth containing hydrolyzed soya proteins, extracts of Hypericum perforatum, extracts of Hamamelis virginiana, extracts of Arnica montana and extracts of Salix alba and, in addition, urea, menthol, propylene glycol and salicylic acid.

In the context of the invention, the term “plant” applies both to whole plants and to parts of plants (leaves, roots, flowers, bark) and mixtures thereof. The extracts differ in composition according to the starting material selected and the method used for extraction.

A preferred embodiment of the invention is the use of the above-described compositions containing the plant extracts mentioned, the Hamamelis virginiana extracts preferably emanating from the leaves of the plant, the Arnica montana extracts preferably emanating from the flowers and the Salix alba extracts preferably emanating from the bark.

The use of compositions containing the described synergistic mixtures has, above all, the advantage that the growth of healthy hair is reduced, i.e. there is no painful or aggressive treatment of the skin so that here, too, there are no wounds or dermal irritation to contend with. Only hair growth is inhibited to the point where it can be completely stopped.

Besides their inhibiting effect on hair growth, the synergistic mixtures can also have astringent, toning, soothing, refreshing and wound-healing properties.

Soya Proteins

The soya proteins are preferably obtained from soya flour by extraction with demineralized water. For hydrolysis, the aqueous solution of the proteins is enzymatically hydrolyzed with proteases. In principle, any proteases with a hydrolyzing effect in either a basic or alkaline medium are suitable. According to the invention, the proteins are hydrolyzed at least once in a basic medium, preferably at pH 8.7, and at least once in an acidic medium, preferably at pH 3.6. The pH values can differ according to the enzyme. The reaction temperature for the hydrolysis is between 20 and 80° C., preferably between 30 and 60° C. and more particularly 54° C.

In the context of the invention, the term “plant” applies both to whole plants and to parts of plants (leaves, roots, flowers) and mixtures thereof.

Hypericum perforatum

The plant Hypericum perforatum L. (Hypericaceae) is also known as Saint-John's-wort and belongs to the family of Guttiferae. It is a widespread herbaceous plant with golden-yellow panticulated inflorescences. The plant contains 1% essential oil with alpha-pinene, monoterpenes and n-alkanes and the flavonoids quercetin, its 3-galactoside (hyperin) and rutin and also quercetin and isoquercetin. The leaves collected at flowering time are medicinally used as tea or in the form of tinctures obtained therefrom and the clear deep red oil obtained from the fresh flowers which contains ca. 0.1% hypericin.

Hamamelis virginiana

The plant Hamamelis virginiana is a North American shrub (Hamamelidaceae) which produces yellow flowers in late fall and which is also known as witch hazel. The plant is also cultivated in Europe. The leaves and bark contain tannins, the leaves additionally containing flavone glycosides and essential oils. Hamamelis water, a distillate of hamamelis leaves and branches, has a toning effect on the skin mainly through its content of essential oils. The highly colored extracts contain tannins—typically the hamamelis tannins, galloyl esters which give gallic acid and hamamelose (C6H12O6, MR180,16), a branched-chain sugar, on hydrolysis. According to the invention, the use of extracts from the leaves of the plant is preferred.

Hamamelis preparations are used to staunch relatively minor bleeding and against varices, hemorrhoids, hematomas, varicose veins, excoriation, itching, burns and frostbite. Applied internally, hamamelis extract—like other tannin-containing drugs—is active against diarrhea.

Arnica montana

Arnica montana is a herb of dry meadows of the Subalpine to Alpine region with orange-yellow flowers having a faintly spicy perfume and a slightly bitter taste. Arnica montana contains 0.2-0.4% essential oil, bitter principles, particularly sesquiterpene lactones (helenalin and derivatives), flavone glycosides (astragalin, isoquercetin). According to the invention, the use of extracts from the flower of the plant is preferred.

Oily extracts (arnica oils) and alcohol extracts (arnica tincture) obtained from arnica flowers or roots and arnica-containing ointments are externally applied in the treatment of bruises, hematomas, etc. by virtue of their circulation-promoting effect. Internally, arnica has a favorable effect against gastrointestinal problems and inflammation of the mouth and throat. In concentrated form, however, it irritates the skin and mucous membrane.

Salix alba

The genus Salix, dioecious trees or bushes, is widespread in the Northern Hemisphere and is divided into around 500 species. The Salix species are also known as willows and the bark of these willows is stripped from two- to three-year-old branches and contains 1 to 12% salicin or salicylalcohol derivatives, other phenolic compounds and 8 to 20% tannins. It represents so to speak a “prodrug” for salicylic acid and was used accordingly against fever, rheumatic pain, headache and inflammation. On account of its relatively poor compatibility, willow bark has been displaced as a therapeutic agent by acetyl salicylic acid or arylacetic acids (for example Diclofenac, Ibuprofen).

According to the invention, the use of extracts from the bark of the plant is preferred.

Extraction

The extracts to be used in accordance with the invention are prepared by known methods of extracting plants or parts thereof. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found, for example, in Hagers Handbuch der pharmazeutischen Praxis (5th Edition, Vol. 2, pp. 1026-1030, Springer Verlag, Berlin-Heidelberg-New York 1991). Fresh or dried plants or parts thereof are suitable as the starting material although plants and/or plant parts which may be mechanically size-reduced and optionally defatted before extraction are normally used. Any size reduction methods known to the expert, for example comminution with a bladed tool, may be used.

Preferred solvents for the extraction process are water, organic solvents or mixtures of organic solvents and water, more particularly propylene glycol or low molecular weight alcohols, esters, ethers, ketones or halogenated hydrocarbons with more or less large water contents (distilled or non-distilled), preferably aqueous alcoholic solutions with more or less large water contents. Extraction with distilled water, methanol, ethanol, propanol, butanol and isomers thereof, acetone, propylene glycols, polyethylene glycols, ethyl acetate, dichloromethane, trichloromethane and mixtures thereof, more particularly a mixture of distilled water and propylene glycol, is particularly preferred. The extraction process is generally carried out at 20 to 100° C. and preferably at 20 to 80° C. In one possible embodiment, the extraction process is carried out in an inert gas atmosphere to avoid oxidation of the ingredients of the extract. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients, for example by filtration. The extraction process may be carried out to any degree, but is usually continued to exhaustion. The present invention includes the observation that the extraction conditions and the yields of the final extracts may be selected according to the desired application. If desired, the extracts may then be subjected, for example, to spray drying or freeze drying. The quantity of plant extracts used in the preparations mentioned is governed by the concentration of the individual ingredients and by the way in which the extracts are used.

In the context of the present invention, the term “extract” or “plant extract” applies both to dried extracts and to mixtures of dried extracts with solvent, preferably water, more particularly a mixture of water and propylene glycol.

The use in accordance with the invention of compositions containing the described synergistic mixture for inhibiting hair growth may in principle be used for any cosmetic and/or pharmaceutical preparations used for parts of the body where hair growth is unwanted.

In one particular embodiment of the invention, the compositions are used in deodorants and/or antiperspirants. The above-described synergistic mixture containing hydrolyzed soya proteins and at least one extract of a plant selected from the group consisting of Hypericum perforatum, Hamamelis virginiana, Arnica montana and Salix alba and, more particularly additionally, urea, menthol, propylene glycol and salicylic acid present in deodorants and/or antiperspirants has the required effect for deodorants and/or antiperspirants of inhibiting unwanted hair growth in the armpits besides other effects. Compositions containing a synergistic mixture of hydrolyzed soya proteins, Hypericum perforatum extract, Hamamelis virginiana extract, Arnica montana extract and Salix alba extract and also urea, menthol, propylene glycol and salicylic acid are preferably used for this purpose.

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Germ Inhibitors

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Enzyme Inhibitors

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Odor Absorbers

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexyl-cinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

non-aqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH regulators, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

In another embodiment of the invention, the compositions are used in accordance with the invention in aftershave. The above described synergistic mixture containing hydrolyzed soya proteins and at least one extract of a plant selected from the group consisting of Hypericum perforatum, Hamamelis virginiana, Arnica montana and Salix alba and, more particularly additionally, urea, menthol, propylene glycol and salicylic acid present in the aftershave has the required effect for aftershave of inhibiting unwanted hair growth, particularly beard growth, besides other effects. Compositions containing a synergistic mixture of hydrolyzed soya proteins, Hypericum perforatum extract, Hamamelis virginiana extract, Arnica montana extract and Salix alba extract and also urea, menthol, propylene glycol and salicylic acid are preferably used for this purpose.

Aftershave

The cosmetic preparations which are referred to as aftershave in the present specification and which may be applied after wet or drying shaving include, in principle, any preparations which are applied after shaving, more particularly shaving lotions, aftershave gels and aftershave balm, sprays, foam, creams, sticks, liquid and solid powders. The may be provided with various perfume notes. Aftershaves intended for use after wet or dry shaving are preparations which are supposed to at least sooth irritation of the skin after shaving, to neutralize basic shaving preparations, to re-establish the biological acid covering of the skin and to have a refreshing, cooling and disinfecting effect. Besides the synergistic mixtures, an aftershave may be made up of glycerin (or glycol derivatives), citric acid, alum, disinfectant, perfumes and alcohol. A product sprayable from spray cans is obtained, for example, by adding 85% alcohol, perfume, polyvinyl pyrrolidone and gaseous propellants.

The many possible uses according to the invention of compositions containing a synergistic mixture for inhibiting hair growth containing hydrolyzed soya proteins and at least one extract of a plant selected from the group consisting of Hypericum perforatum, Hamamelis virginiana, Arnica montana and Salix alba and, more particularly additionally, urea, menthol, propylene glycol and salicylic acid are very attractive both to the market and to the consumer. Accordingly, the complex problem addressed by the invention has been solved by the use of these compositions.

A preferred embodiment of the invention is the use of the compositions described above, the components present in the synergistic mixture in the compositions having the following composition:

(a) 0.01 to 40% by weight hydrolyzed proteins from soya extract, preferably from 0.01 to 36.3% by weight, from 0.1 to 10% by weight, from 0.7 to 7.3% by weight, more preferably from 1.5 to 3.6% by weight and more particularly 3.63% by weight

(a) 0.005 to 10% by weight Hypericum perforatum extract, preferably from 0.01 to 7% by weight, from 0.07 to 3.5% by weight, more preferably from 0.35 to 0.7 and more particularly 0.35% by weight and/or

(a) 0.005 to 10% by weight Hamamelis virginiana extract, preferably from 0.01 to 6% by weight, from 0.06 to 3% by weight, more preferably from 0.3 to 0.7 and more particularly 0.3% by weight and/or

(b) 0.005 to 10% by weight Arnica montana extract, preferably from 0.01 to 6% by weight, from 0.06 to 3% by weight, more preferably from 0.3 to 0.7 and more particularly 0.3% by weight and/or

(c) 0.001 to 10% by weight Salix alba extract, preferably from 0.005 to 3% by weight, from 0.01 to 1.5% by weight, more preferably from 0.15 to 0.3 and more particularly 0.15% by weight and optionally

(d) 0.0005 to 10% by weight menthol, preferably from 0.005 to 1% by weight, from 0.01 to 0.5% by weight, more preferably from 0.05 to 0.1 and more particularly 0.05% by weight and

(e) 0.0005 to 10% by weight urea, preferably from 0.01 to 4% by weight, from 0.04 to 2.0% by weight, more preferably from 0.02 to 0.28 and more particularly 0.2% by weight and

(f) 0.005 to 40% by weight propylene glycol, preferably from 0.1 to 35% by weight, from 0.3 to 25% by weight, more preferably from 1 to 10 and more particularly 1.5% by weight and

(g) 0.0005 to 3% by weight salicylic acid, preferably from 0.001 to 0.25% by weight, from 0.0025 to 0.125% by weight, more preferably from 0.0125 to 0.025 and more particularly 0.0125% by weight,

with the proviso that the quantities shown add up to 100% by weight optionally with water and/or other auxiliaries and additives.

The components may be used as dry matter or in solution. In the case of the hydrolyzed proteins from soya extract, it is preferred to use aqueous solutions, more particularly aqueous solutions with a dry matter content of 8 to 10% by weight and preferably 9% by weight and a protein content of 3 to 5% by weight and preferably 4% by weight.

The Hypericum perforatum, Hamamelis virginiana, Arnica montana and Salix alba extracts may be used as dry matter or in solution. Both possibilities are referred to an extracts in the context of the present invention. They are preferably used in solution. Preferred solvents are water and/or a water/propylene glycol mixture. The ratio of water to propylene glycol may be between 1:1.5 and 2:1 and is preferably 1:1. The dry matter content of the extracts to be used is between 1 and 5% by weight, preferably between 1.5 and 3% by weight and more particularly 2% by weight.

A preferred embodiment of the invention is the use of the above-described compositions, the synergistic mixture having the following composition:

72.75% by weight hydrolyzed soya proteins

7% by weight Hypericum perforatum extract

6% by weight Hamamelis virginiana extract and

6% by weight Arnica montana extract and

3% by weight Salix alba extract and

4% by weight urea and

1% by weight menthol and

0.25% by weight salicylic acid

The use of hydrolyzed soya proteins preferably corresponds to an aqueous solution which has a protein content of 4% and a dry matter content of 9%. The Hypericum perforatum extracts are preferably used in solution, the solvent consisting of water and propylene glycol. A mixture of equal parts of water and propylene glycol is preferred. The percentage dry matter content is 2% by weight.

The hamamelis virginiana extracts are preferably used in solution, the solvent consisting of water and propylene glycol. A mixture of equal parts of water and propylene glycol is preferred. The percentage dry matter content is 2% by weight.

The Arnica montana extracts are preferably used in solution, the solvent consisting of water and propylene glycol. A mixture of equal parts of water and propylene glycol is preferred.

The Salix alba extracts are preferably used in solution, the solvent consisting of water and propylene glycol. A mixture of equal parts of water and propylene glycol is preferred. The percentage dry matter content is 1.5% by weight.

The propylene glycol content of the mixture is from 25 to 40% by weight.

This mixture is preferably incorporated in cosmetic preparations in a quantity of 5% by weight at temperatures below 50° C. The mixture is soluble in water but insoluble in fats and oils.

The extracts according to the invention have an active substance content in the extracts of 1 to 100% by weight, preferably 10 to 95% by weight and more particularly 20 to 80% by weight. In the context of the invention, the active substance content is understood to be the sum of all the active substances present in the extract, based on the dry weight of the extract.

Active substance in the context of the invention relates to the ingredients present in the extract, even if their content and identity cannot be established by conventional methods known to the expert. Active substances in the context of the invention are also understood to be any ingredients present in the extract of which the effect is already known or of which the effect cannot yet be established by conventional methods known to the expert.

Active substance in the context of the invention relates to the percentage of substances and auxiliaries and additives present in the composition except for the water additionally introduced.

The total percentage content of auxiliaries and additives can be from 1 to 50% by weight and is preferably from 5 to 40% by weight, based on the final preparation of the cosmetic and/or dermatological preparations. The preparations may be produced by standard cold or hot methods.

The present invention includes the observation that particularly effective cosmetic and/or pharmaceutical preparations are obtained by the co-operation of the ingredients of the synergistic mixture. They have an excellent skin-care effect and high dermatological compatibility. They also show high stability, particularly against oxidative decomposition of the products.

The terms “preparations”, “final preparations” and “compositions” are synonymous in the context of the present invention.

The compositions containing a synergistic mixture that inhibits hair growth may be used for the production of cosmetic and/or pharmaceutical preparations such as, for example, sprays, creams, gels, lotions, alcohol and water/alcohol solutions, emulsions, wax/fat compounds, stick preparations, powders or ointments. These preparations may additionally contain mild surfactants, oil components, emulsifiers, pearlizing waxes, consistency factors, thickeners, superfatting agents, stabilizers, polymers, silicone compounds, fats, waxes, UV protection factors and antioxidants, lecithins, phospholipids, biogenic agents, film formers, swelling agents, self-tanning agents, tyrosine inhibitors (depigmenting agents), hydrotropes, solubilizers, preservatives, perfume oils, dyes and the like as further auxiliaries and additives.

Surfactants

Suitable surfactants are anionic, nonionic, cationic and/or amphoteric or zwitterionic surfactants which may be present in the compositions in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols (cf. DE 197 56 377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates such as, for example, Dicaprylyl Carbonate (Cetiol® CC), Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Dicaprylyl Ether (Cetiol® OE), ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Emulsifiers

Suitable emulsifiers are, for example, nonionic surfactants from at least one of the following groups:

products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and alkylamines containing 8 to 22 carbon atoms in the alkyl group;

alkyl and/or alkenyl oligoglycosides containing 8 to 22 carbon atoms in the alk(en)yl group and ethoxylated analogs thereof;

addition products of 1 to 15 mol ethylene oxide onto castor oil and/or hydrogenated castor oil;

addition products of 15 to 60 mol ethylene oxide onto castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 mol ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 mol ethylene oxide;

mixed esters of pentaerythritol, fatty acids, citric acid and fatty alcohol according to DE 1165574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethyleneglycol-30 Dipolyhydroxystearate;

polymer emulsifiers, for example Pemulen types (TR-1, TR-2) of Goodrich;

polyalkylene glycols and

glycerol carbonate.

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as refatting agents for cosmetic formulations from DE 2024051 PS.

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide with the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide with the sorbitan esters mentioned are also suitable.

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryidimonium Hydroxypropyl Hydrolyzed Collagen (Lamequat® L, Grünau), quaternized wheat polypeptides, polyethyleneimine, cationic silicone polymers such as, for example, Amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar® CBS, Jaguar® C-17, Jaguar® C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosm. Toil. 108, 95 (1993).

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

UV Protection Factors and Antioxidants

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)-camphor as described in EP 0693471 B1;

4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably 4-methoxybenzalmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB);

propane-1,3-diones such as, for example, 1-(4-tert.butylphenyl)-3-(4′-methoxyphenyl)-propane-1,3-dione;

ketotricyclo(5.2.1.0)decane derivatives as described in EP 0694521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 19712033 A1 (BASF). The UV-A and UV-B filters may of course also be used in the form of mixtures. Particularly favorable combinations consist of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used. Other suitable UV filters can be found in P. Finkel's review in SÖFW-Journal 122, 543 (1996) and in Parf. Kosm. 3, 11 (1999).

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Biogenic Agents

Biogenic agents in the context of the invention are additionally those which do not originate from the described plants such as, for example, tocopherol acetate, tocopherol palmitate, ascorbic acid, (deoxy)ribonucleic acid and fragmentation products thereof, retinol, bisabolol, allantoin, phytantriol, panthenol, AHA acids, ceramides, pseudoceramides, essential oils, other plant extracts and additional vitamin complexes.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Self-Tanning Agents and Depigmenting Agents

A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Perfume Oils

Suitable perfume oils are mixtures of natural and synthetic fragrances. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Dyes

Suitable dyes are any of the substances suitable and approved for cosmetic purposes as listed, for example, in the publication “Kosmetische Färbemittel” of the Farbstoffkommission der Deutschen Forschungs-gemeinschaft, Verlag Chemie, Weinheim, 1984, pages 81 to 106. These dyes are normally used in concentrations of 0.001 to 0.1% by weight, based on the mixture as a whole.

### EXAMPLES

### 1. Example

Preparation of the Hamamelis Extract

2 kg dried Hamamelis virginiana extract were dissolved with stirring at room temperature in a mixture of 48 kg distilled water and 49 kg propylene glycol and then filtered. The dried extract had been purchased.

### 2. Example

Extraction of Arnica montana Plants

10 kg Arnica montana flowers were extracted with stirring at room temperature by maceration for 48 hours in a mixture of 44 kg distilled water and 45 kg propylene glycol. The extraction mixture was then filtered.

### 3. Example

Preparation of the Hypericum perforatum Extract

2 kg dried Hypericum perforatum extract were dissolved with stirring at room temperature in a mixture of 48 kg distilled water and 49 kg propylene glycol. The solution was then filtered. The dried extract had been purchased.

### 4. Example

Preparation of the Salix alba Extract

15 kg willow (Salix alba) bark were extracted for 3 hours at 80° C. in a mixture of 66 kg distilled water and 66 kg propylene glycol. The solution was then filtered.

5. Preparation of Hydrolyzed Soya Proteins

15 kg soya flour were extracted with distilled water for 4 hours at 55° C. The extraction mixture was then centrifuged. For hydrolysis, the product was first enzymatically hydrolyzed with a protease for 4 hours at 54° C./pH 8.7 and then for 160 mins. at 54° C./pH 3.6. To inactivate the enzymes, the mixture was heated for one hour to 95° C. and the protein mixture obtained was filtered. The solution contained 9% by weight dry matter and 4% by weight proteins.

6. Composition of the Synergistic Mixture

The synergistic mixture added to the compositions for the uses according to the invention has, for example and preferably, the following composition (figures=% by weight). This mixture is commercially obtainable, for example, under the name of Pilinhib® VEG (a trade mark of the Cognis Group):

| | |
|---|---|
| hydrolyzed soya proteins | 72.75% |
| Hypericum perforatum extract | 7.00% |
| Hamamelis virginiana extract | 6.00% |
| Arnica montana flower extract | 6.00% |
| urea | 4.00% |
| Salix alba bark extract | 3.00% |
| menthol | 1.00% |
| salicylic acid | 0.25% |

This mixture contains inter alia between 25 and 40% by weight propylene glycol (from the water/propylene glycol solvent) from the Hypericum perforatum, Hamamelis virginiana, Arnica montana and Salix alba extracts.

In the interests of simplicity, the name Pilinhib® VEG is used in the following Examples for mixtures having the above-mentioned composition.

7. Preparation of the Synergistic Mixture

The menthol and the salicylic acid were dissolved in propylene glycol at 50° C. All other components were added with stirring at room temperature. The mixture was then centrifuged and filtered.

8. Test for Determining the Inhibition of Hair Growth

A deodorant lotion containing 5% by weight Pilinhib® VEG was tested against a placebo (deodorant without Pilinhib® VEG) on the armpits of 12 healthy male and female volunteers aged between 18 and 50 years. All the volunteers naturally had vigorous and rapid underarm hair growth.

Before the actual treatment, the underarm hair growth expected after 10 days was determined. To this end, the armpits of the volunteers were shaved and, after 10 days, the hair length and hair diameter was determined by macrophotography.

To determine their hair growth inhibiting properties, the products were applied twice a day (mornings and evenings) for 10 days. Before the treatment, both armpits were shaved. For direct comparison, one armpit was treated with the lotion containing 5% by weight Pilinhib® VEG and the other armpit with the lotion containing no Pilinhib® VEG. After the ten-day treatment, the hair length and hair diameter were determined by macrophotography.

TABLE 1
Determination of hair length and hair diameter

| | D20/D10 |
|---|---|
| Deodorant containing 5% by weight | 42% |
| Pilinhib ® VEG | |
| Placebo deodorant | 0% |

D10 = 10 days after shaving without the treatment
D20 = 10 days after shaving with the treatment

After only 10 days' treatment, the hair length and hair diameter of the volunteers were reduced by an average of 42% by comparison with the non-treatment. In the treatment with placebo, no change in hair length was observed in relation to the non-treatment.

1. The use of a composition containing a synergistically active mixture which inhibits hair growth and which contains hydrolyzed soya proteins and at least one extract of a plant selected from the group consisting of Hypericum perforatum, Hamamelis virginiana, Arnica montana and Salix alba.

2. The use claimed in claim 1, characterized in that the synergistic mixture additionally contains substances selected from the group consisting of urea, menthol, propylene glycol and salicylic acid.

3. The use claimed in claim 1 and/or 2, characterized in that the Hamamelis virginiana extracts preferably emanate from the leaves of the plant, the Arnica montana extracts preferably emanate from the flowers and the Salix alba extracts preferably emanate from the bark.

4. The use of the composition claimed in any of claims 1 to 3 as a deodorant and/or antiperspirant.

5. The use of the composition claimed in any of claims 1 to 3 as an aftershave.

6. The use claimed in any of claims 1 to 5, characterized in that the components present in the synergistic mixture in the compositions claimed in claims 1 to 5 have the following composition:
(c) 0.01 to 40% by weight hydrolyzed proteins from soya extract
(d) 0.005 to 10% by weight Hypericum perforatum extract
(h) 0.005 to 10% by weight Hamamelis virginiana extract
(i) 0.005 to 10% by weight Arnica montana extract
(j) 0.001 to 10% by weight Salix alba extract
(k) 0.0005 to 10% by weight menthol
(l) 0.0005 to 10% by weight urea
(m) 0.005 to 40% by weight propylene glycol
(n) 0.0005 to 3% by weight salicylic acid
with the proviso that the quantities shown add up to 100% by weight optionally with water and/or other auxiliaries and additives.

7. The use claimed in any of claims 1 to 5, characterized in that the synergistic mixture has the following composition:
72.75% by weight hydrolyzed soya proteins
7% by weight Hypericum perforatum extract
6% by weight Hamamelis virginiana extract and
6% by weight Arnica montana extract and
3% by weight Salix alba extract and
4% by weight urea and
1% by weight menthol and
0.25% by weight salicylic acid.
